(19) United States
(12) Patent Application Publication
(10) Pub. No.: US 2026/0002938 A1
(43) Pub. Date: Jan. 1, 2026

(54) KIT FOR EARLY DIAGNOSIS AND MONITORING OF TUMORS

(71) Applicant: ROMANO LABORATORY SRLS, Lecce (IT)

(72) Inventor: Pietrina Romano, Squinzano (LE) (IT)

(73) Assignee: ROMANO LABORATORY SRLS, Lecce (IT)

(21) Appl. No.: 18/879,876

(22) PCT Filed: Jun. 28, 2023
(86) PCT No.: PCT/IB2023/056680

§ 371 Date: Dec. 30, 2024

(30) Foreign Application Priority Data
Jul. 1, 2022 (IT) 102022000013987

(51) Int. Cl. G01N 33/574; G01N 33/84

(52) U.S. Cl. CPC G01N 33/57407; G01N 33/84; G01N 2800/7028

(57) ABSTRACT

The present invention relates to a ready-to-use kit for the early diagnosis of tumors by the analysis of a sample of a biological liquid and its use for the early diagnosis of tumors.

### BACKGROUND ART

The authors of the report entitled “Global Cancer Statistics 2020”, jointly produced by the American Cancer Society (ACS) and the International Agency for Research on Cancer (IARC), reported that globally one in 5 people will develop a tumor in their lifetime.

According to reports in the pages of CA magazine: In Cancer Journal for Clinicians, where the document was published, in 2020 new tumor cases worldwide were about 19.3 million and deaths caused by the disease about 10 million.

There are prevention programs for different types of cancer. Screening programs are examinations performed on a segment of the population for the purpose of detecting a disease or its precursors, before symptoms or disorders make its presence suspected.

To date, not for all tumors screening tests are available. In fact, a useful examination to ascertain the presence of the disease in the presence of a symptom is not always a valid tool in a screening program, that is, to identify, among the many people who have no disorders, the few who require further investigation. EP3521443 describes a method for the diagnosis of tumors that comprises preparing an extemporaneous solution from a base of iodine and potassium iodide in water and alcohol and mixing it with a saliva sample from a subject to be analyzed. Molecular iodine (I₂) reacts with L-fucose that is optionally present in saliva. L-fucose (6-deoxy-L-galactose) (C₆H₁₂O₅) is the only levogyrous sugar used by mammalian systems that increases in the body under persistent hypoxic conditions when it activates anaerobic glycolysis and the other energy shunt. The increase of L-fucose in the body shows an alteration in the oxidative metabolism of aerobic organisms under various stress conditions, characterized by low oxygen levels (persistent hypoxia) that favor the survival of undifferentiated anaerobic cells.

High levels of serum fucose have been reported in breast cancer, ovarian cancer, lung cancer, liver cancer, thyroid cancer, stomach cancer, pancreatic cancer, head-neck cancer and others.

The method described in EP3521443 has several drawbacks. To begin with, the reagents to be used for the preparation of the solution are not readily available to the normal population. Furthermore, said solution must be kept in the dark and at low temperatures, as it is not stable at all. For these reasons, the described method EP3521443 is not a valid method for performing an early diagnosis of tumors and cannot be put into practice except by individuals with appropriate laboratories and chemical reagents.

Therefore, there is a need for useful tools for the early diagnosis of various types of tumors, which may be put into practice by any individual who wishes to do so, in order to resort to necessary therapies in a timely manner.

### OBJECTS OF THE INVENTION

An object of the invention is to provide a ready-to-use, non-invasive and easy-to-use kit which allows early diagnosis of tumors, in particular but not only breast cancer, ovarian cancer, lung cancer, liver cancer, thyroid cancer, stomach cancer, pancreatic cancer, head-neck cancer and others.

Another object of the invention is to provide a method for early diagnosis of tumors, which comprises the use of the kit of the invention.

A further object of the invention is to provide the use of the kit of the invention for early diagnosis of tumors.

Finally, a further object of the invention is to provide the use of the kit of the invention for monitoring the evolution of cancer disease.

### BRIEF DESCRIPTION OF THE DRAWINGS

FIG. 1 shows an exemplary embodiment of the kit of the invention.

### DESCRIPTION OF THE INVENTION

Starting from the concept proposed by the method described in EP3521443, the Applicant has developed a kit that makes the non-invasive diagnosis of tumors more immediate, easy for anyone, not just for health care professionals or experts in analytical laboratories.

According to one of its aspects, subject-matter of the invention is a kit for the early diagnosis of tumors, comprising:
an optionally graduated container (1), provided with means for its closure (2) and with one or more stirring means (3);
an optionally graduated means for collecting a sample of a biological liquid (4);
a means for transferring a metered amount of sample (5);
a pre-metered amount of molecular iodine (12) (6);
a pre-metered amount of an aqueous iodide solution of an alkali metal (7), preferably potassium iodide;

a slide (8), preferably provided with a cavity;
optionally a colorimetric scale (9); and
optionally an information leaflet (10).

The kit may also comprise an additional container (11) for the initial collection of biological liquid. Alternatively, any clean container may be used to collect the biological liquid.

The container (1) may be, for example, a test tube fitted with a cap (2) made of an appropriate material compatible with iodine, for example, plastic, and is preferably made of dark glass or anyway of a material that does not interact with molecular iodine and iodide.

The container preferably has a capacity of 5 to 20 ml, more preferably 10 to 15 ml, and a suitable thickness to resist shocks to its walls caused by the one or more stirring means (3).

The one or more stirring means (3) may be, for example, small spheres made of glass or other material that does not interact (i.e., does not react) with molecular iodine and potassium iodide.

Preferably, the one or more stirring means are glass spheres, more preferably two or more small glass spheres 2-5 mm in diameter. The presence of the stirring means, in addition to aiding the mixing of reagents with the biological sample, may cause the cells to break down and thus promote the binding of iodine with L-fucose that is optionally present, thus improving the sensitivity of the test. This technical solution was not provided in EP3521443.

The means for collecting a sample of a biological liquid (4) may be graduated, for example, a disposable graduated pipette or a graduated collector of saliva samples, preferably a graduated pipette that may hold 1 to 15 ml of biological liquid, more preferably 1 to 10 ml. Alternatively, the test tube or other container (1) is graduated from 1 to 15 ml or from 1 to 10 ml; in this case, the means for collecting the sample, e.g., a pipette, may collect the sample without measuring it and release it into the test tube or other container until the required volume is reached.

Alternatively, if the means for collecting a sample of a biological liquid (4) is not graduated and neither is the container (1), then there will be a means in the kit for transferring a metered amount of sample (5) into the container (1).

The biological liquid according to the invention is any biological liquid, for example, a sample of saliva, urine, serum or semen, preferably a saliva sample. The pre-metered amount of molecular iodine (12) (6) may range from 1 to 20 mg, preferably 1 to 10 mg, for example, 2, 3, 4, or 5 mg, and is contained in a suitable closed vessel, preferably made of glass or anyway a material that does not interact with molecular iodine. Preferably, molecular iodine is bisublimated iodine.

The iodide solution (7) is preferably potassium iodide. The potassium iodide solution is preferably a 0.5% (w/v) aqueous solution and the pre-metered amount may range from 0.1 ml to 2 ml, preferably from 0.1 to 1 ml, for example, 0.2, 0.3, 0.4 or 0.5 ml, and is contained in a suitable closed vessel preferably made of glass or anyway a material that does not interact with potassium iodide. However, other concentrations of potassium iodide solution may be used in the kit of the invention, taking into consideration the following.

Preferably the solution comprises only water and potassium iodide and does not comprise alcohols, for example, does not comprise ethanol.

The amounts of molecular iodine and potassium iodide solution, as well as its concentration, are correlated. For each 2 mg milligrams of molecular iodine, it is preferable that 0.5 mg of potassium iodide be present in the solution.

Therefore, if the kit contains 4 mg of molecular iodine, the solution may contain 1 mg of potassium iodide.

In other words, the molecular weight ratio of molecular iodine/potassium iodide is about 4/1.

From the amounts given herein for molecular iodine, the skilled in the art is perfectly capable of calculating the amount of iodide salt to be used in the case of using iodide from another alkali metal, for example sodium iodide.

Similarly, the amounts of the two reagents (6) and (7) are correlated to the amount of biological liquid to be analyzed. For each ml of biological liquid, for example saliva, it is preferable to have 2 mg of molecular iodine and 0.5 mg of potassium iodide in aqueous solution in the kit.

Representative examples of proportions of reagents to biological sample are as follows:
2 mg of iodine, 0.5 mg of potassium iodide and 1 ml of biological sample;
4 mg of iodine, 1 mg of potassium iodide and 2 ml of biological sample;
5 mg of iodine, 1.25 mg of potassium iodide and 2.5 ml of biological sample;
6 mg of iodine, 1.5 mg of potassium iodide and 3 ml of biological sample.

The container (1), as well as the vessels containing the reagents (6) and (7), is preferably dark, for example dark glass ampoules, to avoid light transmission inside.

The examples set forth in the experimental section of the present description show representative embodiments of the kit of the invention and the relationships between the biological liquid and the reagents.

The kit of the invention also comprises a slide (8) preferably having a cavity where the sample to be analyzed can be poured in, after the addition of the reagents (6) and (7) and subsequent stirring. The slide is preferably made of glass or anyway of a material that does not interact with the reagents.

The slide may have a colorimetric scale or a stain of one or more reference colors. Alternatively, a colorimetric scale (9) from white to dark brown can be found separately in the kit.

Preferably, the kit may also comprise an information leaflet (10) explaining the procedure for performing the test.

The order of addition of the reagents and biological liquid is not critical. The molecular iodine reagents (6) and iodide solution (7) can be added first and then the biological liquid or vice versa.

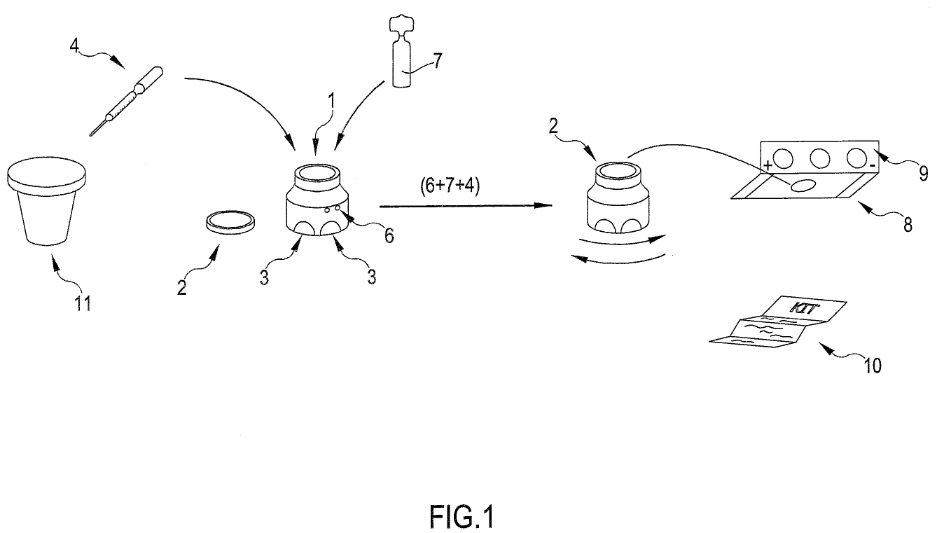

By way of example, according to an embodiment that refers to FIG. 1, to the sample of biological liquid collected from a container (11) by the means for collecting a biological liquid (4), in this case a graduated pipette, poured into the container (1), molecular iodine (6) and iodide solution, preferably potassium iodide solution (7), are added. The color of the sample inside the container turns brown. The container has to be closed and stirred a few seconds, for example 2 to 10 seconds. When stirring is finished, after 1.5-3 minutes a few drops of the treated sample are poured into the slide cavity and verification is done visually, preferably by comparison with the colorimetric scale placed on the slide or separated from the slide.

The test result is positive (presence of tumor) if the color in the container comprising the sample and reagents, initially brown, turns white, a sign that the iodine in the reagents has bound to L-fucose. In the presence of an intermediate color, it is useful to retest after one or more weeks.

The colorimetric scale from white to dark brown can help in evaluating the result, but visual observation alone can also be sufficient.

In contrast to the method described in EP3521443, the kit of the invention is a practical, fast and easy-to-use solution for anyone.

In fact, the perfect pre-measurement of the reagents in the kit and their simple addition into the container where the sample to be examined is placed, is a significant technical advantage over the method of EP3521443.

As mentioned above, the presence of tumors results in increased levels of L-fucose in the body, including biological fluids, such as for example saliva, urine, serum and semen. The reaction between the iodinated reagents and L-fucose results in the color inside the container containing the sample treated according to the invention changing from brown to white.

According to another of its aspects, subject-matter of the invention is the use of the kit for the early diagnosis of tumors, in particular but not only breast cancer, ovarian cancer, lung cancer, liver cancer, thyroid cancer, stomach cancer, pancreatic cancer, head-neck cancer and others.

According to another of its aspects, the invention comprises an in vitro method for the early diagnosis of tumors, in particular but not only breast cancer, ovarian cancer, lung cancer, liver cancer, thyroid cancer, stomach cancer, pancreatic cancer, head-neck cancer and others, which comprises testing a sample of a biological liquid selected from saliva, urine, serum and semen, preferably saliva, with the kit of the invention.

The levels of L-fucose progressively increase as the tumor disease worsens, therefore the kit of the invention is also useful for monitoring the tumor disease, that is, for checking its improvement or worsening.

Thus, according to another of its aspects, subject-matter of the invention is the use of the kit for monitoring the tumor disease, in particular but not only in case of breast cancer, ovarian cancer, lung cancer, liver cancer, thyroid cancer, stomach cancer, pancreatic cancer, head-neck cancer and others.

According to another of its aspects, the invention comprises an in vitro method for monitoring the tumor disease, in particular but not only breast cancer, ovarian cancer, lung cancer, liver cancer, thyroid cancer, stomach cancer, pancreatic cancer, head-neck cancer and others, which comprises testing a sample of a biological liquid selected from saliva, urine, serum and semen, preferably saliva, with the kit of the invention.

The Experimental Section below describes representative embodiments of the invention, for illustrative purposes and in no way limiting.

### EXPERIMENTAL SECTION

### Example 1

A kit according to the invention comprises:
a 10 ml dark glass container equipped with a hard plastic cap and two small glass beads;
a dispensing pipette for collecting the sample of a biological liquid;
4 mg of bisublimated molecular iodine in a closed glass container;
0.2 ml of a 0.5% (w/v) aqueous solution of potassium iodide equivalent to 1 mg of potassium iodide;
a colorimetric scale of white to dark brown; and
an information leaflet.

### Example 2

A kit according to the invention comprises:
a 5 ml amber glass container equipped with a plastic cap and two small glass beads;
a dispensing pipette for collecting the sample of a biological liquid;
2 mg of molecular iodine (I₂) in a closed glass container;
0.1 ml of a 0.5% (w/v) aqueous solution of potassium iodide equivalent to 0.5 mg of potassium iodide;
a slide provided with a cavity and a white to dark brown colorimetric scale on the side; and
an information leaflet.

### Example 3

A kit according to the invention comprises:
a 10 ml dark glass container equipped with a plastic cap and two small glass beads;
a dispensing pipette for collecting the sample of a biological liquid;
6 mg of molecular iodine (I₂) in a closed glass container;
0.3 ml of a 0.5% (w/v) aqueous solution of potassium iodide equivalent to 1.5 mg of potassium iodide;
a slide provided with a cavity;
a colorimetric scale of white to dark brown on the side; and
an information leaflet.

1. A kit for early diagnosis of tumors which comprises:
an optionally graduated container (1), provided with means for its closure (2) and with one or more stirring means (3);
an optionally graduated means for collecting a sample of a biological liquid (4);
a means for transferring a metered amount of sample (5);
a pre-metered amount of molecular iodine (6);
a pre-metered amount of an aqueous iodide solution of an alkali metal (7), preferably potassium iodide;
a slide (8), preferably provided with a cavity;
optionally a colorimetric scale (9); and
optionally an information leaflet (10).

2. The kit according to claim 1, characterized in that said container is a glass test tube provided with a cap and one or more glass beads.

3. The kit according to claim 1 or 2, characterized in that said biological liquid is selected from saliva, urine, serum and semen, preferably saliva

4. The kit according to any one of claims 1 to 3, characterized in that the weight ratio of molecular iodine/potassium iodide is 4/1.

5. The kit according to any one of claims 1 to 4, characterized in that it comprises a slide provided with a cavity.

6. The kit according to any one of claims 1 to 5, characterized in that it comprises a colorimetric scale from white to brown.

7. Use of the kit according to any one of claims 1 to 6, in an in vitro method for early diagnosis of tumors.

8. Use of the kit according to claim 7, wherein said tumor is selected from breast cancer, ovarian cancer, lung cancer, liver cancer, thyroid cancer, stomach cancer, pancreatic cancer, head-neck cancer.

9. Use of the kit according to claim 7 or 8, wherein said sample of a biological liquid is selected from saliva, urine, serum and semen, preferably saliva.

10. An in vitro method for early diagnosis of tumors, particularly but not only, breast cancer, ovarian cancer, lung cancer, and liver cancer, which comprises testing a sample of a biological liquid, preferably saliva, with the kit according to any one of claims 1 to 6.

11. An in vitro method for monitoring the cancer disease, particularly but not only, breast cancer, ovarian cancer, lung cancer, and liver cancer, which comprises testing a sample of a biological liquid, preferably saliva, with the kit according to any one of claims 1 to 6.
